(19) United States
(12) Patent Application Publication
(10) Pub. No.: US 2026/0269321 A1
(43) Pub. Date: Sep. 10, 2026

(54) COMPOUND

(71) Applicant: Sumitomo Chemical Co., Ltd, Tokyo (JP)

(72) Inventors: Florence Bourcet, Godmanchester (GB); Kanika Aggarwal, Godmanchester (GB)

(73) Assignee: Sumitomo Chemical Co., Ltd, Tokyo (JP)

(21) Appl. No.: 19/166,219

(22) PCT Filed: Mar. 20, 2024
(86) PCT No.: PCT/EP2024/057462

§ 371 Date: Sep. 17, 2025

(30) Foreign Application Priority Data
Mar. 24, 2023 (GB) 2304355.7

(51) Int. Cl. H01M 10/0568; C07F 5/04; H01M 10/0525; H01M 10/0569

(52) U.S. Cl. CPC H01M 10/0568; C07F 5/04; H01M 10/0525; H01M 10/0569; H01M 2300/0028

(57) ABSTRACT

A compound of formula (I): wherein: X is Al or B; M⁺ is a metal cation; and R¹ in each occurrence is independently a linear C₃ or C₄ fluorinated alkyl The compound of formula (I) may be used in an electrolyte in a metal or metal ion battery.

### BACKGROUND

WO2022/243470 discloses an electrolyte comprising solvated lithium ions.

WO00/53611 discloses polyfluoroalkoxides for use an electrolyte in batteries and other electrochemical devices.

M Rohde et al, “Li[B(OCH₂CF₃)₄]: Synthesis, Characterization and Electrochemical Application as a Conducting Salt for LiSB Batteries”, Chem Phys Chem Vol 16 (3), 2015, p.666-675 discloses Li[B(OCH₂CF₃)₄] as a water- and thermally stable lithium-ion electrolyte salt for LiS batteries.

EP2827430 discloses compounds of formula (I):

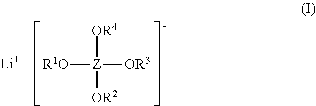

WO2022/161996 discloses an electrolyte based on SO₂ containing at least one conducting salt of formula:

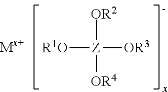

### SUMMARY

The present disclosure provides a compound of formula (I):

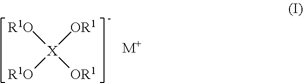

wherein:
X is Al or B;
M⁺ is a metal cation; and
R¹ in each occurrence is independently a linear C₃ or C₄ fluorinated alkyl.

Optionally, at least one R¹ is a linear C₃ alkyl with fewer than 6 fluorine atoms.

Optionally, at least one R¹ contains a group of formula —CF₂H.

Optionally, at least one R¹ is a group of formula —CH₂CF₂CF₂H.

Optionally, X is B.

Optionally, M⁺ is lithium ion.

The present disclosure provides an electrolyte comprising a compound of formula (I) as described herein dissolved in one or more solvents.

The present disclosure provides a metal ion battery comprising an anode, a cathode and an electrolyte as described herein disposed between the anode and the cathode.

The present disclosure provides a metal battery comprising an anode, a cathode and an electrolyte as described herein disposed between the anode and the cathode.

### DESCRIPTION OF DRAWINGS

FIG. 1 is a schematic illustration of a battery comprising a compound as described herein;

and

FIG. 2 shows illustrative initial and steady state Nyquist plots for an illustrative cell;

FIG. 3 is a graph of ionic conductivity vs. solvent: Li+ ratio for cells containing Compound Example 1 and Comparative Compound 1; and

FIG. 4 is a graph of lithium transference number vs. solvent: Li+ ratio for cells containing Compound Example 1 and Comparative Compound 1.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list. References to a layer “over” another layer when used in this application means that the layers may be in direct contact or one or more intervening layers may be present. References to a layer “on” another layer when used in this application means that the layers are in direct contact. References to an element of the Periodic Table include any isotopes of that element.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

### Compounds of Formula (I)

The compound of formula (I) is:

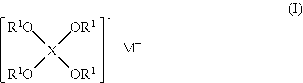

X is Al or B, preferably B.

M⁺ is a metal cation. M⁺ is preferably an alkali metal cation, more preferably Lit.

R¹ in each occurrence is independently a linear C₃ or C₄ fluorinated alkyl.

R¹ in each occurrence is the same or different. Preferably, each R¹ is the same.

The compound of formula (I) may be formed by reacting a compound of formula (II) with one or more of a compound of formula (IIIa) or (IIIb):

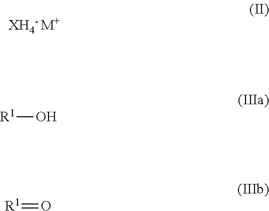

It will be understood that the O atom of formula (IIIa) and of (IIIb) is bound directly to a C atom of R¹.

The compound of formula (IIIa) is a primary alcohol.

The compound of formula (IIIb) is an aldehyde.

Exemplary compounds of formula (II) include, without limitation, lithium aluminium hydride (LiAlH₄) and lithium borohydride (LiBH₄).

### Solvent

An electrolyte containing one or more solvents and a compound of formula (I) may include one or more solvents selected from C₂₋₁₀ alkylene carbonates, di(C₁₋₁₀ alkyl) carbonates, for example propylene carbonate, ethylene carbonate, dimethyl carbonate, diethyl carbonate; linear, branched or cyclic compounds containing two or more ether groups, for example 1,3-dioxolane, 2,5dimethoxy tetrahydrofuran, glyme (dimethoxyethane), diglyme, triglyme and tetraglyme; cyclic lactones and mixtures thereof.

Optionally, a ratio of moles of solvent: moles of M⁺ of the electrolyte is no more than 20:1, optionally less than or equal to 15:1 or less than or equal to 10:1.

### Polymer

An electrolyte as described herein may comprise a polymer, in which case the electrolyte may be a gel.

The polymer may be selected from any known ion-conducting polymers including, without limitation: poly(alkylene oxide), for example poly(ethylene oxide) and poly(propylene oxide); and fluorinated polymers such as PVDF, PVDF-HFP; PMMA; polyacrylonitrile; polycarbonate; polyethylene; polypropylene; poly(vinyl methyl ketone); polyvinylpyrrolidone; polyether ether ketone; polyisoprene; polybutadiene; polystyrene-block-polyisoprene-block-polystyrene; poly(l-vinylpyrrolidone-co-vinyl acetate); polystyrene-block-polybutadiene-block-polystyrene; polystyrene-block-poly(ethylene oxide)-block-polystyrene; and copolymers and mixtures thereof.

The polymer is suitably a neutral polymer, i.e. not a polymer substituted with ionic groups, and in particular is suitably not a single-ion conducting polymer comprising anionic groups.

### Battery

FIG. 1 illustrates a battery comprising a compound of formula (I). The battery may be a metal battery or a metal ion battery, preferably a lithium battery or a lithium-ion battery. The battery is suitably a secondary (rechargeable) battery.

The battery comprises an anode current collector 101 carrying an anode 103 on a surface thereof; a cathode current collector 109 having a cathode 107 disposed on a surface thereof; and a layer 105 comprising an electrolyte comprising a compound as described herein disposed between the anode and the cathode.

The layer 105 may comprise a porous separator in which the electrolyte, e.g., a liquid or gel electrolyte, is absorbed. If the electrolyte comprises a solid, e.g., a gel comprising a polymer and the compound of formula (I), then a porous separator may or may not be present.

Optionally, a ratio of moles of solvent: moles of M⁺ of the electrolyte in the battery is no more than 20:1, optionally less than or equal to 15:1 or less than or equal to 10:1.

In the case of a metal battery, the anode is a layer of metal (e.g., lithium) which is formed over the anode current collector during charging of the battery and which is stripped during discharge of the battery.

In the case of a metal ion battery, e.g., a lithium-ion battery, the anode comprises an active material, e.g., graphite, for absorption of the metal ions.

The cathode may be selected from any cathode known to the skilled person.

The anode and cathode current collectors may be any suitable conductive material known to the skilled person, e.g., one or more layers of metal or metal alloy such as aluminium or copper.

A battery may be formed by providing an electrolyte as described herein on a surface of one of an anode and a cathode and providing the other of an anode and cathode, and associated current collector, over the electrolyte.

A metal battery precursor may be formed by providing an electrolyte as described herein on a surface of an anode current collector; and providing the cathode and a cathode current collector over the electrolyte. Upon application of a charging bias, a metal anode may be formed between the electrolyte and the anode current collector.

FIG. 1 illustrates a battery in which the anode and cathode are separated only by a single layer comprising or consisting of the electrolyte, for example a separator comprising the electrolyte. In other embodiments, one or more further layers may be disposed between the anode and the cathode.

For simplicity, FIG. 1 illustrates a battery in which the anode and cathode are separated only by a single layer 105, however it will be understood that in use a solid-electrolyte interphase will typically form on the anode surface.

A battery as described herein or a battery pack comprising a plurality of batteries as described herein may be used, without limitation, to power electric vehicles or stationary objects.

### Examples

### Electrolyte Example 1

Compound Example 1 was prepared according to the following reaction scheme:

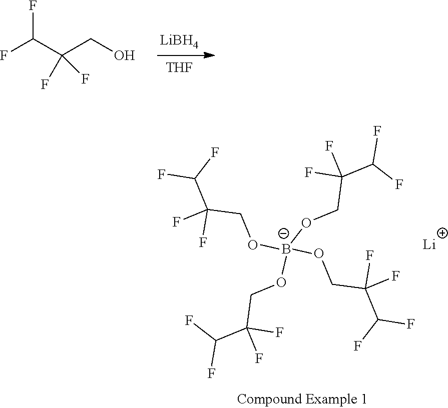 [SMILES: OCC(F)(F)C(F)F ; "LiBH4" ; FC(F)C(F)(F)CO[B-](OCC(F)(F)C(F)F)(OCC(F)(F)C(F)F)OCC(F)(F)C(F)F ; [Li+] ; "THF"]

The reaction was performed under nitrogen atmosphere. A solution of lithium borohydride in tetrahydrofuran (4.2 mL, 8.4 mmol, 2 M) was added drop wise to a solution of 2,2,3,3-tetrafluoropropan-1-ol (3 mL, 33.6 mmol) in 9.30 mL of tetrahydrofuran. The mixture was stirred at room temperature for 1 hour. It was then heated to 65° C. for 1 hour. Additional lithium borohydride (0.45 mL, 0.9 mmol, 2 M in THF) was added drop wise. The mixture was stirred at 65° C. for 1 hour and overnight at room temperature. Propylene carbonate (0.9 mL, 10.9 mmol) was added. The excess solvent was removed under reduced pressure at 30° C. (3.3×10⁻² mbar) for 3 hours to yield a thick transparent oil. Additional propylene carbonate was added to obtain a stable transparent liquid.

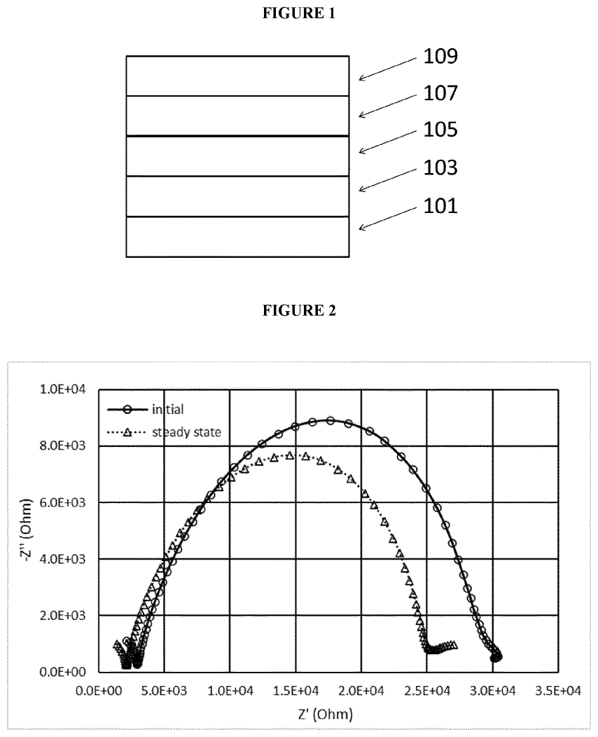

¹H NMR (600 MHz) in deuterated THF (FIG. 2): δ (ppm), 1.38 (d, CH₃, from propylene carbonate 10.08H), 1.77 (m, CH₂, from THF 0.17H), 3.60 (m, CH₂ from THF, 0.18H), 3.76 (t, J=13.88 Hz, 8H), 3.95 (m, CH, from propylene carbonate 3H), 4.48 (t, CH, from propylene carbonate 3H), 4.77 (m, CH, from propylene carbonate 2.66H), 6.12 (tt, J=53.23 Hz, J=5.66 Hz, 4H).

From integration of NMR peaks, it was calculated that for four molecules of TFP in the product electrolyte, which correspond to one lithium cation 3.13 molecules of propylene carbonate was present as residual solvent.

### Coin Cell Examples—General Method

Ionic conductivity and lithium transference number was measured using 2032-type coin cells fabricated in a rigorously dry and oxygen-free Argon gas-filled MBraun glovebox using casings purchased from Cambridge Energy Solutions.

A stainless-steel spacer was inserted in a coin cell bottom, followed by a lithium disk and a fluoro-silicone stencil (purchased from Silex Silicones). The stencil was shaped as a disk of 15.5 mm diameter, with a circular hole of 5 mm diameter cut in its middle (the thickness of the stencil in the crimped cell was 360 μm). The hole was filled with 30ul of electrolyte. On top of the stencil a lithium disk was placed, followed by a stainless-steel spacer, a wave spring and a coin cell top. Finally, the coin cell was crimped.

Electrolyte formulations used to form the coin cells contained either Compound Example 1 or Comparative Compound 1 and a solvent or solvent mixture as set out in Tables 1-4.

Electrolytes containing either Compound Example 1, illustrated above, or Comparative Compound 1, illustrated below.

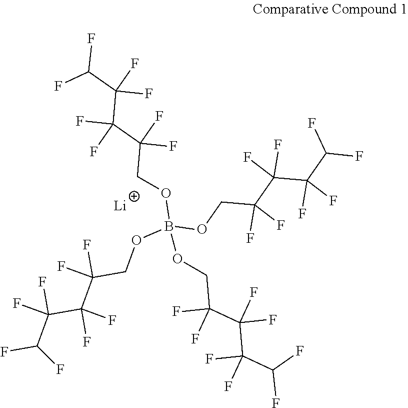

For each compound, cells having different solvent: Li+ molar ratios were made.

### Measurements

Electrochemical Impedance Spectroscopy (EIS) measurements were conducted at room temperature. Electrolyte impedances were taken over a frequency range of 1 Hz to 1 MHz, with an amplitude of 5 mV.

Ionic conductivities were calculated from these data using the following formula:

$$σ = \frac{l}{A \cdot R}$$

where:
1. I is the thickness of the material between the two lithium disks which corresponds to the thickness of the stencil in the crimped cell.
2. A is the area of the film which corresponds to the circular hole cut in the middle of the stencil.
3. R is the electrolyte impedance.

The impedance of the electrolyte is determined by estimating the intercept of the first semicircle of the Nyquist plot with the x-axis. This is the bottom left corner of the illustrative Nyquist plot for a coin cell.

Lithium transference number (LTN) was measured according to Evans's method (J. Evans et al., POLYMER, 1987, Vol 28), using the 2032-type coin cell described above.

Devices were left resting overnight for about 19 hours before performing the LTN measurement, in order to ensure stabilisation of the interfaces between the electrolytes and the lithium disks.

After resting:
1. A first EIS spectrum was measured.
2. This was followed by a DC current measurement (the applied constant voltage is tuned individually for each cell in order to achieve an initial current of about 0.5 uA. The measurement was terminated as soon as the current had decreased to a steady state.
3. The sequence was then finished with a second EIS measurement.

The EIS measurements were conducted at room temperature. The EIS measurements were taken over a frequency range of 1 Hz to 1 MHz, with an amplitude of 5 mV.

The LTN values were calculated according to the following formula, based on the model developed by Evans et al.:

$$LTN = \frac{{I}^{s} ( Δ V - {I}^{0} {R}^{0} )}{{I}^{0} ( Δ V - {I}^{s} {R}^{s} )}$$

where (referring to the illustrative Nyquist plot shown in FIG. 2):
1. R⁰ is the initial impedance taken from the first EIS spectrum (determined by estimating the intercept on the x-axis of the Nyquist plot of the second semicircle (right hand side)).
2. R^{s} is the steady state impedance taken from the second EIS after a DC bias was applied (determined by estimating the intercept on the x-axis of the Nyquist plot of the second semicircle (right hand side)).
3. I⁰ is the initial current taken when the voltage is stepped to set value.
4. I^{s} is the steady state current taken at the end of the DC measurement.

### Results

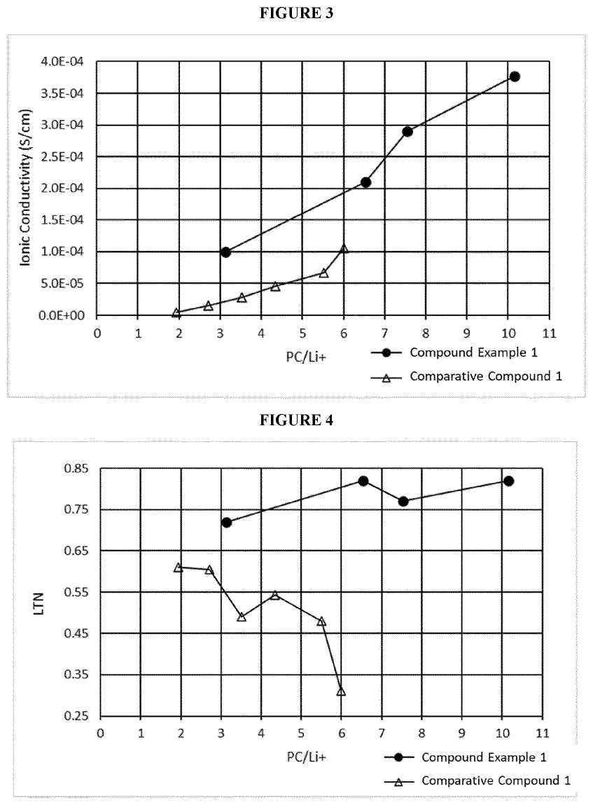

Results are set out in Table 1 for cells containing Compound Example 1 and in Table 2 for cells containing Comparative Compound 1, and in FIGS. 3 and 4.

TABLE 1
Effect of propylene carbonate (PC) on ionic conductivity
and LTN of cells containing Compound Example 1

| | Ionic | |
|---|---|---|
| | conductivity | |
| PC/Li⁺ | [S/cm] | LTN |
| 3.13 | 1.0E−04 | 0.72 |
| 6.54 | 2.1E−04 | 0.82 |
| 7.54 | 2.9E−04 | 0.77 |
| 10.15 | 3.8E−04 | 0.82 |

TABLE 2
Effect of propylene carbonate (PC) on ionic conductivity
and LTN of cells containing Comparative Compound 1

| | Ionic | |
|---|---|---|
| | conductivity | |
| PC/Li⁺ | [S/cm] | LTN |
| 1.9 | 4.4E−06 | 0.61 |
| 2.7 | 1.5E−05 | 0.61 |
| 3.5 | 2.8E−05 | 0.49 |
| 4.3 | 4.6E−05 | 0.54 |
| 5.5 | 6.7E−05 | 0.48 |
| 6.0 | 1.1E−04 | 0.31 |

For both Compound Example 1 and Comparative Compound 1, increasing the PC: Li+ molar ratio results in an increase in ionic conductivity. With reference to FIG. 3, for a given PC: Li+ molar ratio, the ionic conductivity for Compound Example 1 is higher than for Comparative Compound 1.

In the case of Comparative Compound 1, the increase in ionic conductivity is accompanied by a halving of LTN in going from a solvent: Li+ ratio of 2.7:1 to 6.0:1. In contrast, as shown in Table 2 and FIG. 4, surprisingly the LTN for Compound Example 1 is stable across the range of solvent: Li+ molar ratios studied.

1. A compound of formula (I): 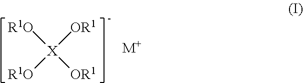
wherein:
X is Al or B;
M⁺ is a metal cation; and
R¹ in each occurrence is independently a linear C₃ or C₄ fluorinated alkyl.

2. The compound according to claim 1 wherein at least one R¹ is a linear C₃ alkyl with fewer than 6 fluorine atoms.

3. The compound according to claim 1 wherein at least one R¹ contains a group of formula —CF₂H.

4. The compound according to claim 1 wherein at least one R¹ is a group of formula —CH₂CF₂CF₂H.

5. The compound according to claim 1 wherein X is B.

6. The compound according to any one of the preceding claim 1 wherein M⁺ is lithium ion.

7. An electrolyte comprising a compound according to claim 1 dissolved in one or more solvents.

8. A metal ion battery comprising an anode, a cathode and an electrolyte according to claim 7 disposed between the anode and the cathode.

9. A metal battery comprising an anode, a cathode and an electrolyte according to claim 7 disposed between the anode and the cathode.
